(19) United States
(12) Patent Application Publication
(10) Pub. No.: US 2010/0282662 A1
(43) Pub. Date: Nov. 11, 2010

(54) APPARATUS FOR PURIFYING BLOOD

(75) Inventors: Kyung Soo Lee, Chungcheongnam-do (KR); Cho Hay Mun, Seoul (KR)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: BHK CO., LTD., Seoul (KR)

(21) Appl. No.: 12/676,738

(22) PCT Filed: Sep. 11, 2008
(86) PCT No.: PCT/KR2008/005373

§ 371 Date: Jul. 27, 2010

(30) Foreign Application Priority Data
Sep. 11, 2007 (KR) 10-2007-0092305
Sep. 10, 2008 (KR) 10-2008-0089411

(51) Int. Cl. A61M 1/34; B01D 15/04; B01D 63/06

(52) U.S. Cl. 210/266

(57) ABSTRACT

The present invention relates to an apparatus for, after separating plasma from blood using a plasma separation filter, purifying the blood by discharging various kinds of toxic substances from the separated plasma using an anion exchange resin filter and a charcoal filter, and more particularly, to an apparatus for purifying blood configured to be capable of miniaturization and be conveniently used by installing the plasma separation filter, the anion exchange resin filter, and the charcoal filter in a housing.

### TECHNICAL FIELD

The present invention relates to an apparatus for, after separating plasma from blood using a plasma separation filter, purifying the blood by discharging various kinds of toxic substances from the separated plasma using an anion exchange resin filter and a charcoal filter, and more particularly, to an apparatus for purifying blood configured to be capable of miniaturization and be conveniently used by installing the plasma separation filter, the anion exchange resin filter, and the charcoal filter in a housing.

### BACKGROUND ART

Generally, a liver is a large organ located in the upper right side of the abdominal cavity, and has the metabolic function of properly processing various nutrients in the body; a function of storing several nutrients needed for the body; a function of secreting of bile necessarily needed to absorb nutrients in bowels; a function of producing albumin, protein, and cholesterol absolutely needed for proper body movements; and a function of counteracting alcohol or medicines or various toxic substances generated from the body.

If the liver function fails, bilirubin is accumulated in the body and jaundice occurs so that the skin and the whites of the eyes of a person are discolored. The person having liver failure may be affected by side effects from medicines since the medicines have not been broken down in the body. The liver is one of the most important internal organs functioning in a broad variety of roles, such as intermediate metabolism, bile secretion, composition, excretion of foreign substances, counteracting poisons, and storing nutrients.

If some parts of the liver are damaged due to a disease, the other parts of the liver can compensate for the impaired functions and it will normally recover following a certain period of time. When it is a serious condition like hepatic insufficiency, a bioartificial liver system is used to artificially revive the liver function.

As the bioartificial liver system currently under clinical evaluation, MARS (Molecular Adsorbent Recirculating System), SPAD (Single Pass Albumin Dialysis), and FPSA (Fractionated Plasma Separation Adsorption) are being used. For the MARS bioartificial liver system, high cost treatment is provided since expensive albumin is used as a dialysis solution and the detoxification is inefficient. The SPAD bioartificial liver system is also a high cost treatment since expensive albumin is used.

The FPSA bioartificial liver system has been proposed in order to reduce the use of expensive albumin, and has a shortcoming of a reliability problem since it is designed such that some portions of the patient's plasma are reinfused into the patient after directly contacting the filter.

A PSAF (Plasma Separation, Adsorption and filtration) bioartificial liver system (Korean Patent No. 0752414), which is configured to eliminate the toxic substances from the plasma without using expensive albumin, was filed and registered by the inventor of the present invention.

The PSAF bioartificial liver system will be described in detail below.

FIG. 1 is a schematic view illustrating the structure of a conventional PSAF bioartificial liver system.

As illustrated in FIG. 1, the conventional PSAF (Plasma Separation, Adsorption and filtration) bioartificial liver system 1 includes a plasma separation filter 10 separating blood into plasma and the blood cells (or blood corpuscles); an adsorption filter 30 filtering the separated plasma with the plasma separation filter 10; a hemofilter 50 eliminating the soluble toxins from the filtered plasma by the absorption filter 30; a replacement fluid supplier 70 replenishing the plasma eliminated the soluble toxins via the hemofilter 50 with replacement fluid; and pumps 90 supplying blood to the plasma separation filter 10 and supplying the plasma separated by the plasma separation filter 10 to the absorption filter 30.

The absorption filter 30 includes an anion exchange resin filter 31 absorbing the negatively charged toxins by the ion exchange mechanism while coupling with the plasma protein such as bilirubin, and a charcoal filter 33 eliminating the toxins coupled with the plasma protein via absorption.

Once a patient's blood is supplied to the plasma separation filter 30, the plasma will be separated by the plasma separation filter 30, and the toxins like bilirubin will be eliminated from the separated plasma by flowing into the anion exchange filter 31. After that, the separated plasma will entered the charcoal filter 33 and the toxins like tryptophan will be removed from the separated plasma and the soluble toxins will be discharged by the hemofilter 50.

If the conventional PSAF bioartificial liver system is used, the treatment expense can be reduced since the soluble toxic substances and the protein-coupled toxins can be eliminated from the plasma only by using a small amount of plasma replacement fluid without using expensive albumin; and the plasma will be quickly purified using two absorption filters and a hemofilter including the anion exchange filter and the charcoal filter.

### DISCLOSURE OF INVENTION

### Technical Problem

However, the plasma separation filter, the anion exchange filter, and the charcoal filter included in the conventional PSAF bioartificial liver system are configured to filter the fluid by flowing it in the longitudinal direction like the general fluid filtration filter, and as such the plasma separation filter, the anion filter, and the charcoal filter must be arranged in the longitudinal direction. The size of the whole system becomes bigger since the space for the laying out of each filter needs to be ensured and there is a shortcoming with the problems of the system installation and the setting since each filter needs to be arranged in an accurate order respectively.

The present invention has been proposed to solve the aforementioned problems, and embodiments of the present invention provide an apparatus for purifying blood constructed to be easily installed and used by realizing the miniaturization of the apparatus by means of unifying each filter.

### Technical Solution

In an exemplary embodiment of the present invention, the apparatus for purifying blood includes a plasma separation filter separating plasma from blood and an absorption filter filtering the plasma, wherein the absorption filter is coupled to cover the plasma separation filter.

The plasma separation filter may allow, when blood is flowing into one inner side, the plasma to separately pass through a side wall and blood cells to be discharged through the other inner side. The plasma separation filter may include an internal diaphragm inserted in between the plasma separation filter and the absorption filter to cover the side wall of the plasma separation filter, wherein the internal diaphragm has an internal diaphragm through-hole in a side wall thereof through which the plasma passes; and a housing coupled to cover the absorption filter, wherein the housing has a plasma outlet in a side wall thereof through which the plasma from the absorption filter is discharged.

The internal diaphragm through-hole and the plasma outlet are formed to be biased towards opposing directions around the axis of the absorption filter.

The internal diaphragm through-hole is formed in a region biased towards a portion where blood enters, and the plasma outlet is formed in a region biased towards a portion where the blood cells are discharged.

A plurality of the internal diaphragm through-holes are formed in order to transversely cover the internal diaphragm.

The outer surface of the internal diaphragm, where the internal diaphragm through-hole is built, is concavely formed.

The absorption filter includes a first absorption filter coupled to cover the internal diaphragm and a second absorption filter coupled to cover the anion exchange resin filter. The absorption filter further includes a middle diaphragm having a middle diaphragm through-hole, for allowing the plasma filtered by the first absorption filter to be delivered to the second absorption filter.

The internal diaphragm through-hole and the middle diaphragm through-hole are biased in opposing directions around the axis of the first absorption filter, and the middle diaphragm through-hole and the plasma outlet are biased in opposing directions around the axis of the second absorption filter.

The internal diaphragm through-hole and the plasma outlet are formed in a region biased towards a portion where blood enters, and the middle diaphragm through-hole is formed in a region biased towards a portion where the plasma is discharged.

A plurality of the internal diaphragm through-holes and a plurality of the middle diaphragm through-holes are formed to transversely cover the inner diaphragm and the middle diaphragm.

The outer surface of the middle diaphragm where the middle diaphragm through-hole is built is concavely formed.

One of the first and second absorption filters is an anion exchange resin filter and the other of the first and second absorption filters is a charcoal filter.

The absorption filter includes a first absorption filter covering one side of opposite longitudinal sides of the internal diaphragm; and a second absorption filter covering the other side of the opposite longitudinal sides of the internal diaphragm, wherein the plasma is allowed, after passing through the plasma separation filter, to sequentially travel the first absorption filter and the second absorption filter.

The plasma separation filter further includes a separation member installed between the first absorption filter and the second absorption filter, wherein the separation member allows particles of both the first absorption filter and the second absorption filter to pass through but does not allow the plasma to pass through.

The separation member includes a plate having at least one plate through-hole and a mesh coupled with the plate to cover the through-hole.

The internal diaphragm through-hole is biased towards the first absorption filter and the plasma outlet is biased towards the second absorption filter.

Each of the plasma separation filter, the internal diaphragm, the absorption filter, and the housing has a cylindrical shape.

The housing includes a body covering an outer side surface of the absorption filter; an inlet cover covering the plasma separation filter and one side of the absorption filter; an outlet cover covering the plasma separation filter and the other side of the absorption filter; a blood inlet formed in the inlet cover for inflow of blood; and a blood cell outlet formed in the outlet cover for discharge of the blood cells.

The housing includes a body covering the outer surface of the side wall of the absorption filter; an inlet cover covering one side of the plasma separation filter and the absorption filter; and an outlet cover covering the other side of the plasma separation filter and the absorption filter. A blood inlet is formed to flow into blood at the inlet cover and a blood cell outlet is formed to discharge the blood cell on the outlet cover.

The inlet cover and the outlet cover have insertion grooves, such that opposite ends of the plasma separation filter and the internal diaphragm are inserted into the insertion grooves, respectively.

In another exemplary embodiment of the invention, the apparatus for purifying blood of the present invention includes a plasma separation filter separating the plasma from blood and an absorption filter filtering the plasma separated from the plasma separation filter are coupled to each other as one body. The absorption filter is configured to direct the plasma flow in a longitudinal direction.

The absorption filter is coupled to cover the plasma separation filter.

At least two of the absorption filters are provided, and two adjacent ones of the absorption filters are arranged to direct the flow of plasma in opposite directions.

At least two of absorption filters are installed, and two adjacent ones of the absorption filters are arranged in series to direct the flow of plasma in the same direction.

### Advantageous Effects

The apparatus for purifying blood of the present invention can be miniaturized by unifying a plasma separation filter and an absorption filter in a body, and increase the convenience of installing and using as well as enhancing the efficiency of the plasma separation filter and the absorption filter.

### BRIEF DESCRIPTION OF THE DRAWINGS

FIG. 1 is a schematic view illustrating the structure of a conventional PSAF bioartificial liver system.

FIG. 2 is a perspective view showing the internal configuration of an embodiment of an apparatus for purifying blood in accordance with the present invention.

FIG. 3 is a cross-sectional view of the embodiment of the apparatus for purifying blood in accordance with the present invention.

FIG. 4 is a cross-sectional view of the usage of the embodiment of the apparatus for purifying blood in accordance with the present invention.

FIG. 5 is a perspective view of an internal diaphragm included in the embodiment of the apparatus for purifying blood in accordance with the present invention.

FIG. 6 is a perspective view of a middle diaphragm included in the embodiment of the apparatus for purifying blood in accordance with the present invention.

FIG. 7 is a vertical sectional view illustrating a coupled configuration of the plasma separation filter and the internal diaphragm included in the embodiment of the apparatus for purifying blood in accordance with the present invention.

FIG. 8 is a cross-sectional view illustrating a coupled configuration of the plasma separation filter and the internal diaphragm included in the embodiment of the apparatus for purifying blood in accordance with the present invention.

FIG. 9 is a perspective view showing the internal configuration of a second embodiment of the apparatus for purifying blood in accordance with the present invention.

FIG. 10 is a cross-sectional view of the second embodiment of the apparatus for purifying blood in accordance with the present invention.

FIG. 11 is a cross-sectional view of an isolation member included in the second embodiment of the apparatus for purifying blood in accordance with the present invention.

FIG. 12 is a cross-sectional view of the usage of the second embodiment of the apparatus for purifying blood in accordance with the present invention.

### MAJOR REFERENCE NUMERALS OF THE DRAWINGS

100: plasma separation filter

200: internal diaphragm

210: internal diaphragm through-hole

300: absorption filter

310: first absorption filter

320: second absorption filter

400: housing 410: body

420: inlet cover 430: outlet cover

500: middle diaphragm

510: middle diaphragm through-hole

610: plate 620: mesh

### BEST MODE FOR CARRYING OUT THE INVENTION

An apparatus for purifying blood in accordance with the present invention is characterized by limiting each filter's shape and installed location, and by restricting the flowing directions of the plasma and the blood cell to specific directions.

Embodiments of the apparatus for purifying blood in accordance with the present invention will be described in detail in conjunction with the accompanying drawings.

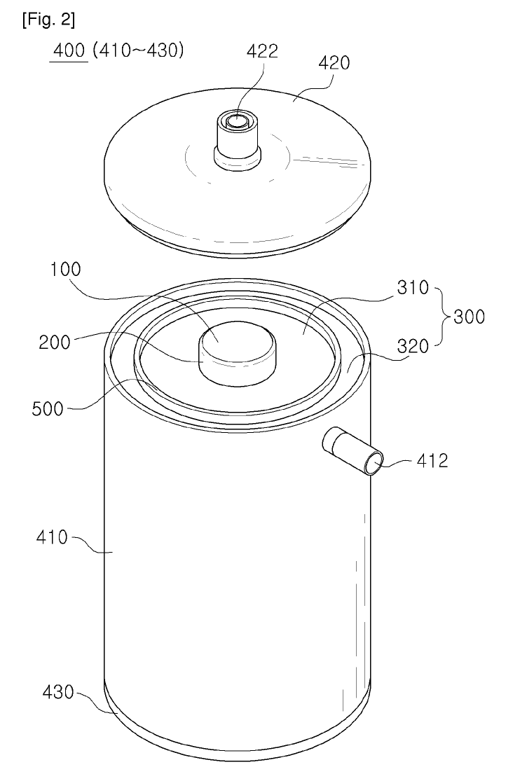

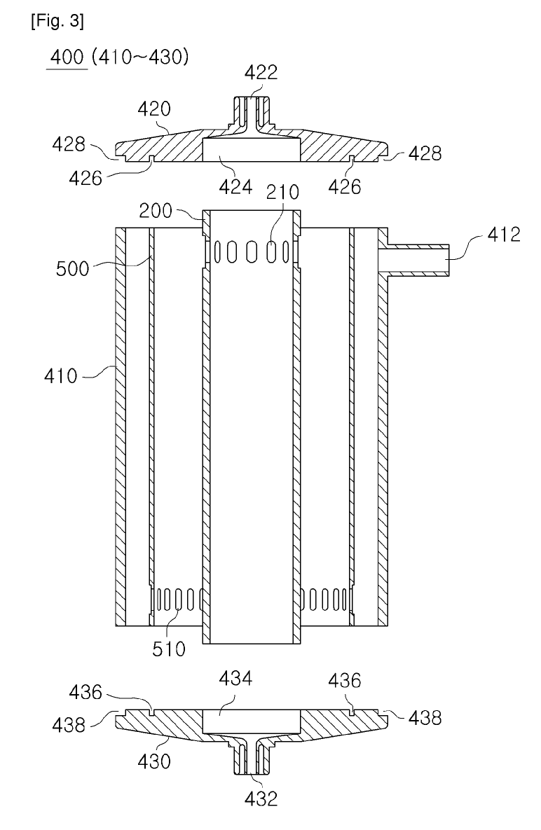

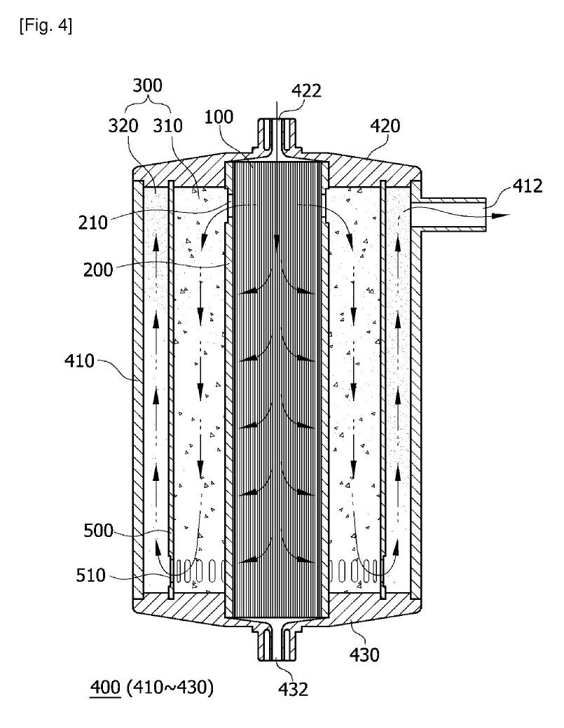

FIG. 2 is a perspective view showing the internal configuration of an embodiment of the apparatus for purifying blood in accordance with the present invention, FIG. 3 is a cross-sectional view illustrating a coupled configuration of a housing and each diaphragm of the embodiment of the apparatus for purifying blood in accordance with the present invention, and FIG. 4 is a cross-sectional view of the usage of the embodiment of the apparatus for purifying blood in accordance with the present invention.

The apparatus for purifying blood in accordance with the present invention includes a plasma separation filter 100 constructed to allow plasma to separately pass through a side wall and blood cells to be discharged to the other side (the bottom side in this embodiment) when blood is flowing into an inner side (the upper side in this embodiment); an internal diaphragm 200 coupled to cover the side wall of the plasma separation filter 100 and formed with an internal diaphragm through-hole 210 on the side wall; an absorption filter 300, coupled to cover the internal diaphragm 200, for filtering the discharged plasma via its passage through the internal diaphragm through-hole 210; and a housing coupled to cover the absorption filter 300 and formed with a plasma outlet 412 on the side wall in order to discharge the filtered plasma outside.

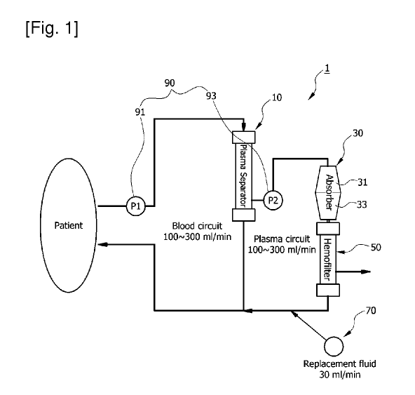

When compared to the conventional plasma separation filter 10 and the absorption filter 30 illustrated in FIG. 1, the plasma separation filter 100 and the absorption filter 300 are different in shape and the coupling configuration, and detailed description thereof will be omitted since the basic principles of separating the blood into the plasma and the blood cells are the same.

Although the plasma separation filter 100 separating the plasma from blood and the absorption filter 300 filtering the plasma are generally constructed to flow the blood and the plasma in the longitudinal direction, the plasma separation filter 10 and the absorption filter 30 applied to the conventional bioartificial liver system as shown in FIG. 1 had to be arranged in series since they are separately manufactured. Consequently, a user using the conventional bioartificial liver system experiences a lot of inconveniences when it comes to installing the plasma separation filter 10 and the absorption filter 300 respectively according to the proper order and securing enough space for installing the plasma separation filter 10 and the absorption filter 30.

However, the plasma separation filter 100 and the absorption filter 300 applied to the present invention are coupled to be reciprocally piled one upon the other, in other words, since the plasma separation filter 100 and the absorption filter 300 can be unified into one body by coupling the absorption filter 300 to cover the side surface of the plasma separation filter 100, the user can install the plasma separation filter 100 and the absorption filter very easily without thinking of the orders of the plasma separation filter 100 and the absorption filter 300. If the plasma separation filter 100 and the absorption filter 300 are manufactured as one body, the numbers of the components and the installation space can be considerably reduced because an extra tube connecting the plasma separation filter 100 with the absorption filter 300 is not needed.

If the plasma separation filter 100 is constructed to directly contact the absorption filter 300, there is a worry about the separated plasma flowing in the transverse direction (horizontal direction in this embodiment) of the absorption filter 300 instead of flowing in the longitudinal direction of the absorption filter 300 while the separated plasma by the plasma separation filter 100 is passing through the absorption filter 300. In the embodiment of the apparatus for purifying blood in accordance with the present invention, the internal diaphragm is additionally formed between the plasma separation filter 100 and the absorption filter 300 in order to guide the flow direction of the separated plasma by the plasma separation filter 100 to pass through the absorption filter 300 in the longitudinal direction because the absorption filter 300 cannot be efficiently used since the area in contact with the absorption filter 300 becomes narrower when the plasma is flowing in the transverse direction of the absorption filter 300.

An internal diaphragm through-hole 210 is formed in the internal diaphragm to allow discharged plasma from the plasma separation filter 100 to flow into the absorption filter 300 and the internal diaphragm through-hole 210 can be formed to be biased towards one side of the internal diaphragm 200 in order to maximize the area over which the plasma contacts the absorption filter 300. The internal diaphragm through-hole 210 can be formed to be biased towards where blood is flowing in (upper portion in this embodiment) since a large amount of plasma will be discharged to one side surface of the plasma separation filter 100 when blood is entering one side of the plasma separation filter 100.

The internal diaphragm 200 and the middle diaphragm 500 can be omitted in the embodiment of the apparatus for purifying blood in accordance with the present invention when the plasma is entering only one side between two longitudinal sides of the absorption filter 300 and is discharged only through the other side.

The absorption filter 300 can be formed as one body and also can be constructed with a first absorption filter 310 and a second absorption filter 320 which perform the different functions as in the embodiment.

For example, when an anion exchange resin filter, coupled with the plasma protein, for absorbing bilirubin, and a charcoal filter, coupled with the plasma protein, for absorbing tryptophan, are needed, the first absorption filter 310 can be applied to the anion exchange resin filter and the second absorption filter 320 can be applied to the charcoal filter. Only the case where the first absorption filter 310 is applied to the exchange resin filter and the second absorption filter 320 is applied to the charcoal filter is described in this embodiment, the first absorption filter 310 and the second absorption filter 320 can be applied to various kinds of filters depending on a variety of situations such as the blood condition, the purifying condition, and the like.

When the absorption filter 300 is constructed as the first absorption filter 310 and the second absorption filter 320 as in the aforementioned case, the middle diaphragm 500 is formed between the first absorption filter 310 and the second absorption filter 320 to allow the plasma to smoothly pass through the first absorption filter and the second absorption filter 320 in the longitudinal direction.

When the middle diaphragm 500 is installed, a middle diaphragm through-hole 310 is formed in the middle diaphragm 500 to allow the separated plasma by the plasma separation filter 100 to pass through the first absorption filter 310 and the second absorption filter 320 step by step, the middle diaphragm through-hole 510 is biased towards the other side of the internal diaphragm through-hole 210 to allow the plasma transferred to the first absorption filter 310 via the internal diaphragm through-hole 210 to be transferred to the second absorption filter 320 after passing through the first absorption filter 310 in the longitudinal direction. When the internal diaphragm through-hole 210 is formed on the top of the internal diaphragm 200 as illustrated in this embodiment, the middle diaphragm through-hole 510 can be formed at the bottom of the middle diaphragm 500.

A plasma outlet 412 installed on the side surface of the body 410 is formed to be biased towards the other side of the middle diaphragm through-hole 510 to allow the transferred plasma to the second absorption filter 320 via the middle diaphragm through-hole 510 to be discharged outside of the housing 400 after passing through the second absorption filter 320 in the longitudinal direction. When the internal diaphragm through-hole 210 is formed in the top portion (i.e., the area biased towards the area where blood is flowing in) of the internal diaphragm 200, the middle diaphragm through-hole 510 can be formed in the bottom portion of the middle diaphragm 500 (i.e., the area biased towards the area where the blood cell is discharging) and the plasma outlet 412 can be formed in the top portion (i.e., the area biased towards the area where blood is flowing in) of the body 410.

When the absorption filter 300 is constructed as one filter instead of separating into the first absorption filter 310 and the second absorption filter 320, in other words, the middle diaphragm 500 is excluded, the plasma outlet 412 needs to be formed at the opposite side (i.e., the area towards the area where the blood cell is biased) of the internal diaphragm through-hole 210.

The housing 400 includes the body 410 formed as a cylinder shape to cover the outside surface of the absorption filter 300; an inlet cover 420 covering one side (i.e., the top portion in this embodiment) of the plasma separation filter 100 and the absorption filter 300; and an outlet cover 430 covering the other side (i.e., the bottom portion in this embodiment) of the plasma separation filter 100 and the absorption filter 300. At one side of the body 410, the plasma outlet 412 is formed in order to discharge the filtered plasma by the absorption filter 300, a blood inlet 422 is formed for the inflow of blood at the inlet cover 420, and a blood cell outlet 432 is formed for the discharge of the blood cell at the outlet cover 430.

When the inlet cover 420 and the outlet cover 430 are constructed to simply cover both ends of the plasma separation filter 100 and the internal diaphragm 200, there is a concern that the blood, which entered the blood inlet 422, can flow into the absorption filter 300 without passing through the plasma separation filter 100; when the inlet cover 420 and the outlet cover 430 are constructed to simply cover both sides of the middle diaphragm 500, there is a concern that the first absorption filter 310 and the second absorption filter 320 cannot be surely separated; when the inlet cover 420 and the outlet cover 430 are constructed to simply cover both sides of the body 410, there is a concern that the plasma can be drained between the body 410 and the inlet cover 420 or between the body 410 and the outlet cover 430. Therefore, at the inlet cover 420 and the outlet cover 430 as illustrated in FIG. 3, plasma separation filter grooves 424 and 434 for the insertion of both sides of the plasma separation filter 100 and the internal diaphragm 200, middle diaphragm grooves 426 and 436 for the insertion of both sides of the middle diaphragm 500, and body accepting grooves 428 and 438 for the insertion of both sides of the body 410 can be formed respectively.

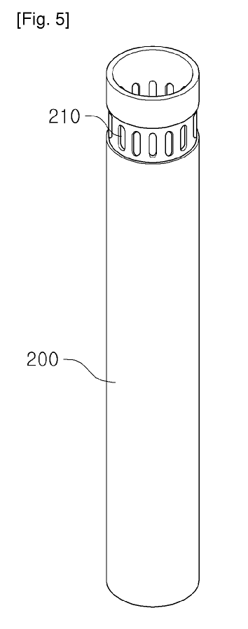

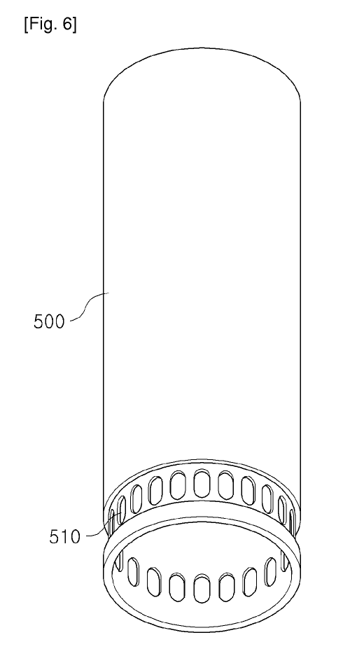

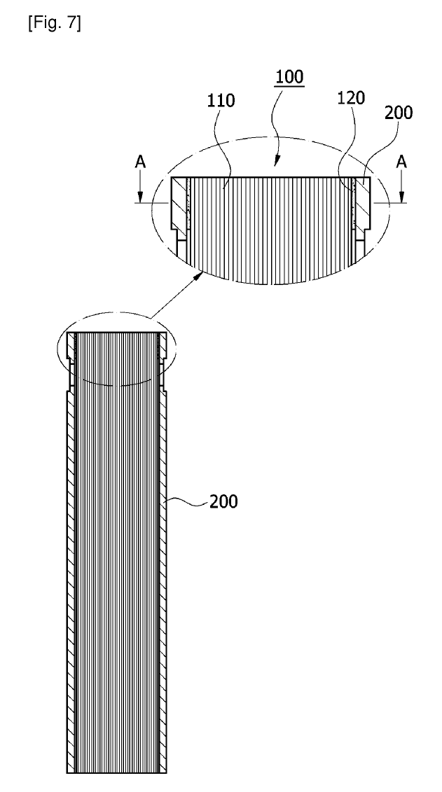

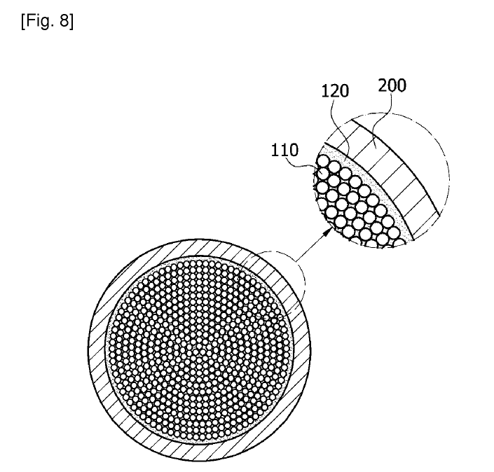

FIG. 5 is a perspective view of an internal diaphragm included in the embodiment of the apparatus for purifying blood in accordance with the present invention, FIG. 6 is a perspective view of a middle diaphragm included in the embodiment of the apparatus for purifying blood in accordance with the present invention, FIG. 7 is a vertical sectional view illustrating a coupled configuration of the plasma separation filter and the internal diaphragm included in the embodiment of the apparatus for purifying blood in accordance with the present invention, and FIG. 8 is a cross-sectional view taken along the line A-A of FIG. 7, illustrating a coupled configuration of the plasma separation filter and the internal diaphragm included in the embodiment of the apparatus for purifying blood in accordance with the present invention.

If the internal diaphragm through-hole 210 is formed only in one side of the internal diaphragm 200, the efficiency of the first absorption filter 310 will be much lowered since the plasma separated by the plasma separation filter 100 cannot be evenly distributed to each portion. In the same manner, if the middle diaphragm through-hole 510 is formed only in one side of the middle diaphragm 500, the efficiency of the second absorption filter 320 will be much lower since the plasma passed through the first absorption filter 310 cannot be evenly distributed to each portion.

To resolve the aforementioned problems, the internal diaphragm through-hole 210 and the middle diaphragm through-hole 510 are formed with multiple numbers to transversely cover the internal diaphragm 200 and the middle diaphragm 500 as illustrated in FIGS. 5 and 6. The number and the cross-sectional area of the internal diaphragm through-hole 210 and the middle diaphragm through-hole 510 can be properly changed depending on the inflow volume of the separated plasma by the plasma separation filter 100 and the efficiency of the absorption filter 300.

Furthermore, if the area where the internal diaphragm through-hole 210 and the middle diaphragm through-hole 510 are formed is manufactured with the same thickness as the other area, there is a concern that the plasma can be delivered only to the corresponding portion with the internal diaphragm through-hole 210 and the middle diaphragm through-hole 510 instead of evenly being delivered to the whole internal circumference of the first absorption filter 310 and the second absorption filter 320 since the first absorption filter 310 and the second absorption filter 320 are pressing the internal diaphragm through-hole 210 or the middle diaphragm through-hole 510 with great force. Therefore, the internal diaphragm 200 and the middle diaphragm 500 are constructed in a concaved shape at the outer surface area where the internal diaphragm through-hole 210 and the middle diaphragm through-hole 510 are formed, and so that the flowed plasma via the internal diaphragm through-hole 210 and the middle diaphragm through-hole 510 can be evenly delivered to the whole internal circumference of the first absorption filter 310 and the second absorption filter 320.

When the outer surface area, where the internal diaphragm through-hole 210 and the middle diaphragm through-hole 510 are formed, are constructed in a concave shape along the outer surface of the internal diaphragm 200 and the middle diaphragm 500, the efficiency of the first absorption filter 310 and the second absorption filter 320 can be improved since the plasma discharged via the internal diaphragm through-hole 210 and the middle diaphragm through-hole 510 can flow into the arranged direction of the internal diaphragm through-hole 210 and the middle diaphragm through-hole 510 to a predetermined extent along the concave-shaped portion.

If the internal diaphragm 200 and the middle diaphragm 500 can pull the plasma separation filter 100 and the first absorption filter 310 inside, they can be applied to the polygonal pipe or the non-radially shaped pipe; and a round-shape pipe or a cylinder shape can be preferred to allow the plasma to evenly flow into all the internal diaphragm through-hole 210 and all the middle diaphragm through-hole 510. When the internal diaphragm 200 and the middle diaphragm 500 are formed in cylindrical shapes, the plasma separation filter 100, the absorption filter 300, and the body 410 also need to be formed in a cylindrical shape.

The plasma separation filter 100 included in the present invention is constructed with a bunch of tiny hollow fibers 110 as shown in FIGS. 7 and 8. The hollow fibers 110 are constructed to allow the plasma to be discharged by passing through the side wall and the blood cell to be discharged to the other side along the internal path when blood is flowing into the inner side. Because the structure of the plasma separation filter 100 formed with a plurality of hollow fibers 100 is actually the same as the conventional membrane filter, more detailed description is omitted.

When a space is secured in between the hollow fibers 110 and the internal diaphragm 200, and in between different hollow fibers 100, the blood, which entered one side of the plasma separation filter 100 in the longitudinal direction, flows through the spaces between the hollow fibers 110 and the internal diaphragm 200 and the space between the hollow fibers 110 and the different hollow fibers instead of only flowing inside of the hollow fibers 110, so that the efficiency of the plasma separation is drastically lowered.

Therefore, binding materials are inserted to fill the spaces between the hollow fibers 110 and the internal diaphragm 200 and the spaces between the different fibers 110 on both longitudinal sides (top and bottom portion in FIG. 7) of the plasma separation filter 100. The binding materials 120 have the liquidity when the binding materials 120 are injected to the space between the hollow fibers 110 and the internal diaphragm 200; and they will be changed to the solid shape and seal tight the space between the hollow fibers 110 and the internal diaphragm 200 and the spaces between the different fibers 110 after some period of time has elapsed.

Once the binding materials 120 are installed on both longitudinal sides of the plasma separation filter 100, all the blood, which entered one side of the plasma separation filter 100 in the longitudinal direction, flows into the inside of the hollow fibers 110 and so that the efficiency of the plasma separation filter can be highly improved.

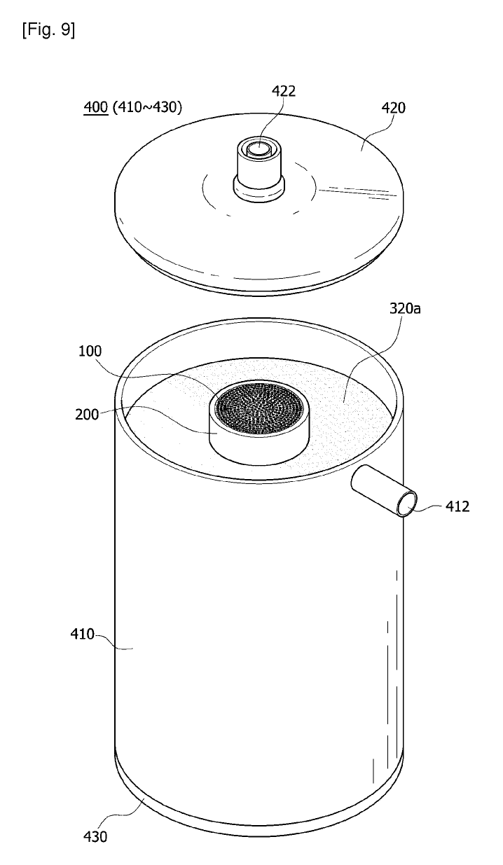

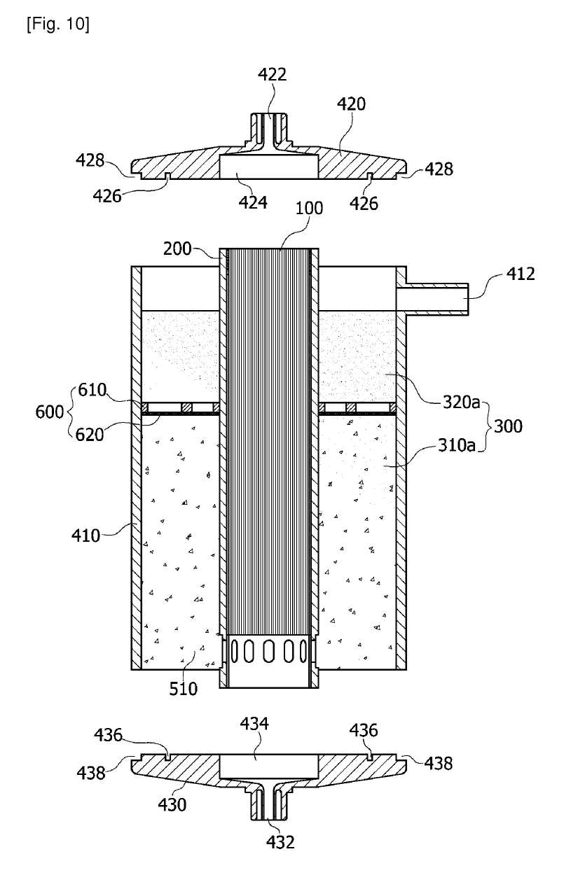

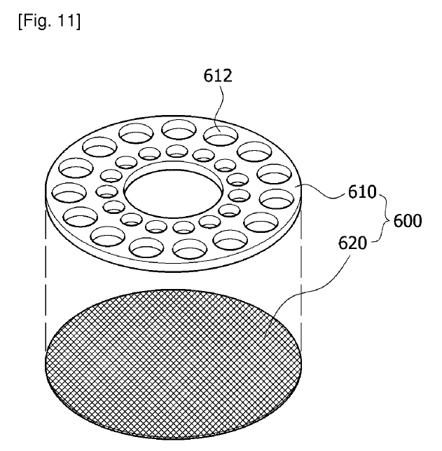

FIG. 9 is a perspective view showing the internal configuration of a second embodiment of the apparatus for purifying blood in accordance with the present invention, FIG. 10 is a cross-sectional view of the second embodiment of the apparatus for purifying blood in accordance with the present invention, and FIG. 11 is a cross-sectional view of an isolation member included in the second embodiment of the apparatus for purifying blood in accordance with the present invention.

In this embodiment of the apparatus for purifying blood in accordance with the present invention, the first absorption filter 310a and the second absorption filter 320a are constructed to be arranged in series according to the inflow direction of the plasma. The first absorption filter 310a can be arranged to cover one side (i.e., the bottom side of the internal diaphragm 200 in this embodiment) of opposite longitudinal sides of the internal diaphragm 200 and the second absorption filter 320a can be arranged to cover the other (i.e., the top side of the internal diaphragm 200 in this embodiment) of the opposite longitudinal sides of the internal diaphragm 200.

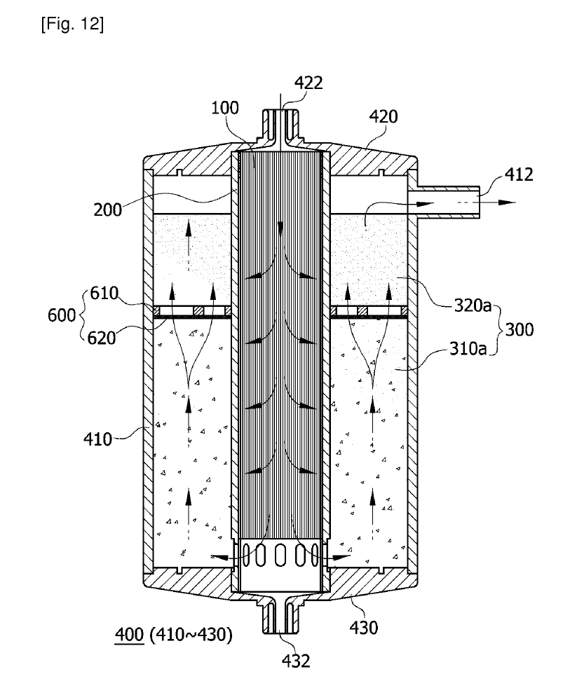

When the first absorption filter 310a and the second absorption filter 320a are arranged according to the inflow direction of the plasma, the plasma passed through the plasma separation filter 100 can travel through the first absorption filter 310a and the second absorption filter 320a step-by-step. The advantages and the effects gained from the plasma's passing through the first absorption filter 310a and the second absorption filter 320a step-by-step will be described in detail below with reference to FIG. 12.

Between the first absorption filter 310a and the second absorption filter 320a, a separation member 600 is installed to allow only the plasma to pass through while the substances of the first absorption filter 310a and the substances of the second absorption filter 320a cannot pass through. With the separation member 600, the first absorption filter 310a is separated from of the second absorption filter 320a.

The separation member 600 includes a plate 610 formed with at least one plate through-hole 612 and a mesh 620 coupled with the plate 610 to cover the through-hole. The mesh is very densely formed and plays a role not to allow the particle of the first absorption filter 310a and the particle of the second absorption filter to pass through; and the plate 610 plays a role to support the mesh 620 so as not to be distorted or changed.

If there is no concern that the mesh 620 be distorted or changed without being supported by the plate, the separation member can be formed with the mesh 620 only.

The internal diaphragm through-hole 210 is formed to be biased towards the first absorption filter 310a and the plasma outlet 412 is formed to be biased towards the second absorption filter 320a in order to allow the supplied blood via the internal diaphragm through-hole 210 after being separated by the plasma separation filter 100 to pass through the first absorption filter 310a and the second absorption filter 320a consecutively.

Furthermore, the internal diaphragm through-hole 210 can be built at the corresponding portion with the bottom of the first absorption filter 310a in order to allow the transferred plasma flowing through the first absorption filter 310a via the internal diaphragm through-hole 210 to be delivered to the second absorption filter 320a after passing through the first absorption filter in the longitudinal direction; and the plasma outlet 412 formed at the side wall of the body can be built at a predetermined portion corresponding to the top portion of the second absorption filter 320a in order to allow the transferred plasma to the second absorption filter 320a via the separation member 600 to be discharged outside of the housing after passing through the second absorption filter 320a in the longitudinal direction.

FIG. 12 is a cross-sectional view of the usage of the second embodiment of the apparatus for purifying blood in accordance with the present invention.

When the second embodiment of the apparatus for purifying blood of the present invention is used, the plasma separated by the plasma separation filter 100 can be delivered to the first absorption filter 310a via the internal diaphragm through-hole 210 formed at the bottom of the internal diaphragm 200. At this time, the plasma separated by the plasma separation filter 100 can be collected at the bottom because of its own weight. When the internal diaphragm through-hole 210 is formed at the bottom of the internal diaphragm 200, the plasma can be more efficiently delivered to the first absorption filter 310a.

The plasma, which entered the bottom of the first absorption filter 310a, moves upwardly and can be discharged outside of the housing 400 via the plasma outlet 412 after passing through the separation member 600 and the second absorption filter 320a consecutively.

In case of the apparatus for purifying blood with the configuration illustrated in FIG. 4, since the plasma separated by the plasma separation filter 100 will enter the first absorption filter 310 by moving up, and flow into the second absorption filter 320 by moving down, and then be discharged outside of the housing 400 via the plasma outlet 412 by moving up again, the inflow direction of the plasma changes 180 changes three times. When the apparatus for purifying blood with the configuration illustrated in FIG. 4 is used, the purifying efficiency can be lowered because the plasma cannot flow smoothly.

On the other hand, in case of the apparatus for purifying blood with the configuration illustrated in FIG. 12, since the inflow direction of the plasma changes 180 degrees only one time, the plasma can flow smoothly and consequently the purifying efficiency can be increased.

While the present invention has been described in connection with the exemplary embodiments, it is not to be limited thereto but will be defined by the appended claims. It is to be understood that those skilled in the art can substitute, change or modify the embodiments in various forms without departing from the scope and spirit of the present invention.

1. An apparatus for purifying blood, wherein the apparatus comprises:
a plasma separation filter (100) which separates plasma from the blood; and
an absorption filter (300) which filters the plasma, wherein the absorption filter (300) is coupled to cover the plasma separation filter (100).

2. The apparatus of claim 1, wherein the plasma separation filter (100) allows, when the blood is flowing into one inner side of the plasma separation filter (100), the plasma to separately pass through a side wall of the plasma separation filter (100) and blood cells to be discharged through another inner side of the plasma separation filter (100), and includes:
an internal diaphragm (200) inserted in between the plasma separation filter (100) and the absorption filter (300) to cover the side wall of the plasma separation filter (100),
wherein the internal diaphragm (200) has an internal diaphragm through-hole (210) in a side wall thereof through which the plasma passes; and
a housing (400) coupled to cover the absorption filter (300), wherein the housing (400) has a plasma outlet (412) in a side wall thereof through which the plasma from the absorption filter (300) is discharged.

3. The apparatus of claim 2, wherein the internal diaphragm through-hole (210) and the plasma outlet (412) are formed to be biased in different directions around the axis of the absorption filter (300).

4. The apparatus of claim 3, wherein
the internal diaphragm through-hole (210) is formed in a region biased towards a portion where the blood enters, and
the plasma outlet (412) is formed in a region biased towards a portion where the blood cells are discharged.

5. The apparatus of claim 2, wherein a plurality of the internal diaphragm through-holes (210) are formed in order to transversely cover the internal diaphragm (200).

6. The apparatus of claim 2, wherein an outer surface of the internal diaphragm (200) where the internal diaphragm through-hole (210) is built is concavely formed.

7. The apparatus of claim 2, wherein the absorption filter (300) includes:
a first absorption filter (310) covering the internal diaphragm (200); and
a second absorption filter (320) covering the first absorption filter (310).

8. The apparatus of claim 7, further comprising a middle diaphragm (500) having a middle diaphragm through-hole (510), wherein the middle diaphragm through-hole (510) allows the plasma filtered by the first absorption filter (310) to be delivered to the second absorption filter (320).

9. The apparatus of claim 8, wherein
the internal diaphragm through-hole (210) and the middle diaphragm through-hole (510) are biased in opposite directions around the longitudinal axis of the first absorption filter (310), and wherein
the middle diaphragm through-hole (510) and the plasma outlet (412) are biased in opposite directions around the longitudinal axis of the second absorption filter (320).

10. The apparatus of claim 9, wherein
the internal diaphragm through-hole (210) and the plasma outlet (412) are formed in a region biased towards a portion where the blood enters; and
the middle diaphragm through-hole (510) is formed in a region biased towards a portion where the plasma is discharged.

11. The apparatus of claim 8, wherein a plurality of the internal diaphragm through-holes (210) and a plurality of the middle diaphragm through-holes (510) are formed to transversely cover the inner diaphragm (200) and the middle diaphragm (500).

12. The apparatus of claim 8, wherein an outer surface of the middle diaphragm (500) where the middle diaphragm through-hole (510) is built is concavely formed.

13. The apparatus of claim 7, wherein one of the first and second absorption filters (310, 320) is an anion exchange resin filter and the other one of the first and second absorption filters (310, 320) is a charcoal filter.

14. The apparatus of claim 2, wherein the absorption filter (300) includes:
a first absorption filter (310a) covering one side of opposite longitudinal sides of the internal diaphragm (200); and
a second absorption filter (320a) covering the other side of the opposite longitudinal sides of the internal diaphragm (200),
wherein the plasma is allowed, after passing through the plasma separation filter (100), to sequentially travel the first absorption filter (310a) and the second absorption filter (320a).

15. The apparatus of claim 14, further comprising a separation member installed between the first absorption filter (310a) and the second absorption filter (320a), wherein the separation member allows particles of both the first absorption filter (310a) and the second absorption filter (320a) to pass through but does not allow the plasma to pass through.

16. The apparatus of claim 15, wherein the separation member (600) includes:
a plate (610) having at least one plate through-hole (612); and
a mesh (620) coupled with the plate (610) to cover the through-hole (612).

17. The apparatus of claim 14, wherein the internal diaphragm through-hole (210) is biased towards the first absorption filter (310a) and the plasma outlet (412) is biased towards the second absorption filter (320a).

18. The apparatus of claim 14, wherein one of the first and second absorption filters (310, 320) is an anion exchange resin filter and the other one of the first and second absorption filters (310, 320) is a charcoal filter.

19. The apparatus of claim 2, wherein each of the plasma separation filter (100), the internal diaphragm (200), the absorption filter (300), and the housing (400) has a cylindrical shape.

20. The apparatus of claim 2, wherein the housing (400) includes:
a body (410) covering an outer side surface of the absorption filter (300);
an inlet cover (420) covering the plasma separation filter (100) and one side of the absorption filter (300);
an outlet cover (430) covering the plasma separation filter (100) and another side of the absorption filter (300);
a blood inlet (422) formed in the inlet cover (420) for inflow of blood; and
a blood cell outlet (432) formed in the outlet cover (430) for discharge of the blood cells.

21. The apparatus of claim 20, wherein the inlet cover (420) and the outlet cover (430) have insertion grooves (424, 434), such that opposite ends of the plasma separation filter (100) and the internal diaphragm (200) are inserted into the insertion grooves (424, 434), respectively.

22. An apparatus for purifying blood, wherein said apparatus comprises:
a plasma separation filter (100) separating plasma from the blood; and
an absorption filter (300) filtering the plasma separated by the separation filter (100),
wherein the plasma separation filter (100) and the absorption filter (300) are coupled to each other as one body, and
wherein the absorption filter (300) is configured to direct a plasma flow in a longitudinal direction.

23. The apparatus of claim 22, wherein the absorption filter (300) is coupled to cover the plasma separation filter (100).

24. The apparatus of claim 22, wherein
at least two absorption filters (300) are provided, and
two adjacent absorption filters (310, 320) are arranged to direct the plasma flow in opposite directions.

25. The apparatus of claim 22, wherein
at least two absorption filters (300) are installed, and
two adjacent absorption filters (310a, 320a) are arranged in series to direct the plasma flow in a same direction.
